(19) United States
(12) Patent Application Publication
(10) Pub. No.: US 2025/0367609 A1
(43) Pub. Date: Dec. 4, 2025

(54) Atomic Scale Paired Electron Bonding, Adsorption and Chelation for Removing Preservative Biocidal Agents from Rx and OTC Ophthalmic Therapeutics and Other Medical Solutions

(71) Applicants: Terry Earl Brady, The Valley (AI); Anthony Lee Dellinger, Burlington, NC (US); Abed Alqader Ibrahim, Greensboro, NC (US); Christopher E. Starr, New York, NY (US); Melinda K. M. Goddard, The Valley (AI); Lowell Hughes, The Valley (AI)

(72) Inventors: Terry Earl Brady, The Valley (AI); Anthony Lee Dellinger, Burlington, NC (US); Abed Alqader Ibrahim, Greensboro, NC (US); Christopher E. Starr, New York, NY (US); Melinda K. M. Goddard, The Valley (AI); Lowell Hughes, The Valley (AI)

(21) Appl. No.: 18/675,650

(22) Filed: May 28, 2024

(51) Int. Cl. B01D 69/14; A61F 9/00; B01D 61/14; B01D 69/02; B01D 71/02; B01D 71/16

(52) U.S. Cl. CPC B01D 69/147; A61F 9/0008; B01D 61/147; B01D 69/02; B01D 71/021; B01D 71/16; B01D 2325/02834; B01D 2325/12

(57) ABSTRACT

Increasing concerns about cytotoxicity caused by traditional ophthalmic product preservatives have driven demand for preservative-free solutions while challenging manufacturers to maintain sterility and safety standards. Inherent user risks have been combined with random manufacturing contamination, resulting in devastating product recalls in recent years. The present invention is a bioactive, affinity filtration system for medical dispensing devices engineered to remove preservatives and contaminants from ophthalmic and other medical solutions when administered. This “just in time” filtration enables manufacturers to follow established preservative practices to ensure safety while delivering a “preservative free” solution upon administration. Without altering the therapeutic formulation, the invention combines adsorption and absorption filtration mechanisms with engineered fullerene derivatives and cellulose acetate membranes to remove preservatives like benzalkonium chloride, thimerosal, and stabilized oxychloro complexes. The invention is designed for integration into established manufacturing, packaging, and stability quality control protocols consistent with the most rigorous antimicrobial patient safety standards.

### FIELD OF THE INVENTION

The present invention relates to medical dispensing devices, specifically to a novel adsorption and chelation-based filter incorporated into dispensers such as dropper bottles, syringes, and spray devices. This filter removes preservatives and contaminants, including organic and inorganic compounds from solutions through different mechanisms, without filtering the active ingredients, immediately prior to administration, thereby maintaining sterility.

### BACKGROUND

The field of ophthalmic sciences involves various professional services, including optometry, ophthalmology, opticianry and pharmacy. This diversity is particularly relevant given the significant market growth in OTC (over-the-counter) eye care products. Ophthalmic diseases often necessitate frequent eye drop applications, introducing risks of contamination from contact with the ocular region, hands, tears and the surrounding environment (Tamrat et al., 2019). These risks led to the inclusion of different preservatives and antimicrobials in most ophthalmic solutions (Feghhi et al., 2008; Taşli and Coşar, 2001; Burdieu et al., 1999; Stevens and Matheson, 1992;Geyer et al., 1995; Fazeli et al., 2004).

Unfortunately, eye care products contaminated during production have also reached consumers in both prescription and OTC formulations while addressing increasing demand. Recent FDA eye drop recalls made national headlines in 2023 and 2024, with the most serious involving Pseudomonas contamination of an OTC lubricating drop (EzriCare®), which resulted in multiple cases of blindness and one death (Mughal and Sakina, 2023).

To mitigate these risks, the most common ocular product additives have included: benzalkonium chloride (BAK), chlorhexidine, polyhexamethylene biguanide (PHMB), stabilized oxychloro complexes (SOCs), sodium perborate and thimerosal (Janulevičienė et al., 2012).

Benzalkonium chloride (BAK), a detergent utilized in 70% of topical ophthalmic solutions, serves as a potent antimicrobial agent, effective against bacteria, fungi and acanthamoeba (Goldstein et al., 2022). The dual hydrophilic and hydrophobic properties of BAK enhance solubility and functionality, disrupting microbial cell walls and leading to cellular lysis. Despite the widespread use and efficacy of BAK, recent concerns over cytotoxic effects have encouraged research into safer alternatives. Studies have shown that minimal inclusions, as low as 0.005%, of BAK in ophthalmic products have the potential to disrupt the corneal barrier, reduce goblet cell density, and destabilize the tear film, all of which contribute to ocular surface toxicity and dry eye symptoms (Walsh and Jones, 2019). Additional adverse effects include inflammation that can lead to decreased vision, discomfort and disrupted mitochondrial function through oxidative stress (Rogov et al., 2020). Notably, the prevalence of ocular surface disease (OSD) is higher in patients with chronic exposure to BAK (Rossi et al., 2013). Efforts remain focused on reducing the inclusion of BAK in eye care products through the use of lower-concentration formulations and BAK-free medications.

Stabilized oxochloro complexes (SOCs) containing chlorite, chlorate, and chlorine dioxide, represent another class of ophthalmic additives that serve as an oxidative preservative and disrupt cellular function by oxidizing cell components, offering strong antimicrobial effects against bacteria and fungi. SOCs break down into natural tear components and are noted for being less cytotoxic to mammalian cells. Notably, comparative research on the inclusion of SOCs in ophthalmic solutions has shown less damage compared to BAK, highlighting its safer profile, however, other studies reported damaged corneal epithelial cells in rabbits. Purite™, known also as a “soft” or “vanishing” preservative in several ophthalmic and cosmetic products, is a common SOC that works by releasing chlorine when exposed to light. This additive can play a key role in maintaining the sterility of the solution while reducing adverse effects. Purite™ thus minimizes the risk of irritation and makes it suitable for users with sensitive and dry eye conditions or those who require frequent product administration.

The use of a proprietary ionic buffered preservative system, like Sofzia™, in various ophthalmic solutions has also proven effective against a wide range of microorganisms. Sofzia™ operates by destabilizing microbial cell membranes, which leads to rapid microbial death, thereby ensuring eye drop safety. Sofzia™ is recognized to be less irritating to the ocular surface compared to stronger preservatives like BAK. While the antimicrobial efficacy and broad-spectrum activity of Sofzia™ are not as potent as BAK, the material has been shown to cause less damage to the corneal epithelium and have less disruption on tear film stability, making it a preferable choice for patients with sensitive eyes and those requiring chronic therapy.

Thimerosal, an early preservative in use since the 1930's, has proven to be highly effective for prevention of microbial contamination in many different multi-dose pharmaceutical formulations. However, the widespread use of thimerosal, an ethylmercury-containing compound, in various medical products including ophthalmic solutions, has sparked concerns over its safety despite the antimicrobial properties. Comprised of 49.55% mercury, thimerosal has long been used as an antimicrobial in a variety of products including topical antiseptics, nasal sprays, and vaccines (Geier et al., 2007). While the United States Centers for Disease Control and Prevention (CDC) has confirmed the safety of thimerosal for use as a flu vaccine preservative (Merthiolate) (Geier et al., 2007), it has been controversial with respect to pediatric exposures through other vaccination protocols since 2000 (Geier and Geier, 2003).

Use of thimerosal specifically in ophthalmic solutions has also been linked to adverse reactions, including conjunctival inflammation, allergic symptoms, ocular discomfort, redness, and irritation of the ocular epithelial cells. Some reactions have been attributed directly to the mercury content, presenting as symptoms of ocular toxicity. Compared to other preservatives, thimerosal has been noted for the compound's broad-spectrum microbiocidal and microbiostatic activity against Gram-positive bacteria, Gram-negative bacteria, yeast and fungi, with little impact on the potency of active pharmaceutical agents (Komatsu et al., 2002). While the use of thimerosal has been largely phased out for ophthalmic solutions in many countries as a result of these adverse effects, the mercuric derivative of thiosalicylic acid remains present in many vaccines, cosmetics, tattoo inks, and disinfectants.

Historically, use of such minimal biocidal agents in aqueous medications was standard until the harmful effects of preservatives, especially on ocular epithelial tissues were fully recognized (Ayaki et al., 2008). In fact, the adverse effects of these biocidal preservatives, including tear film instability, cytotoxicity, and disruption of ocular tissues, can pose significant challenges, particularly in cases of chronic conditions that require long-term treatment (Rosin and Bell, 2013). In an effort to prevent these adverse effects, the industry has increasingly adopted preservative-free medications. Despite the higher cost and inherent user risks for microbial contamination, preservative-free solutions have still failed to fully address sterility issues (Rahman et al., 2006). However, the use of unit-dose droppers has become increasingly common, with one manufacturer adopting a complex design to prevent backflow (which can become increasingly difficult to dispense as the product and air in the vial are evacuated). The single-use packages have also resulted in higher costs, manufacturing complexities, and more environmental waste. The ability to safely reintegrate potent preservative biocidals into these solutions without adverse effects has thus remained an elusive industry challenge.

As a physical alternative to chemical preservatives and antimicrobials, cellulose acetate (CA) has evolved as a basic filtration medium with significant enhancements to meet the stringent requirements of the biomedical industry, particularly in sterility testing and therapeutic administration. As such, CA membranes represent a foundational material associated with the evolution of various membrane filtration technologies, including microfiltration (MF), ultrafiltration (UF), nanofiltration (NF), reverse osmosis (RO), and forward osmosis (FO), addressing a broad spectrum of applications in biomedical fields, water treatment, and food processing to remove contaminants. The utility and filtration capability of CA as a membrane technology is associated with the material's inherent properties, such as biocompatibility, non-toxicity, and film-forming capabilities. The development of CA for use in filtration was directly related to its hydrophilicity and high permeate flux, which are essential for the effective separation of biofluid components and the exclusion of pathogens and macromolecules. Hydrophilic CA membranes are notably effective for use in pressure filtration devices, combining high flow rates, good chemical resistance from pH 4 to 8, and thermal stability with very low adsorption characteristics. These filters are available in various pore sizes and diameters, making them flexible for different applications. Further, the development of biodegradable polymers for filtration membranes highlights the ongoing innovation in membrane science. Cellulose acetate, with its chemical and mechanical stability, is utilized extensively in medical applications and water and wastewater treatment, gas separation and energy generation.

As such, CA has evolved into a highly specialized medium capable of precision separation tasks. Microfiltration membranes have demonstrated significant capabilities in removing a range of contaminants including pathogens, macrometer-sized particles, microscopic bacteria, yeast cells, proteins, and organic colloids. Cellulose acetate has been particularly effective in the construction of MF membranes, utilizing casting solutions comprised of acetone or a combination of acetone and formamide, which aids in achieving the desired porosity and mechanical strength. These characteristics have combined with cost-effectiveness, non-toxic biocompatibility, hydrophilicity, high permeate flux, flexibility and its ease of film formation for a versatile and proven biomedical industry material.

In fact, CA membranes have been used to remove bacteria, viruses, fungi, and an array of particulate contaminants from pharmaceutical products. CA membrane innovations have also included the integration of nanomaterials (or nanofillers) to enhance mechanical strength, thermal stability, filtration efficiency and specificity. The ability to modify the pore size and chemically functionalize the membrane surface has thus led to custom solutions for complex biomedical challenges such as those posed by ensuring sterility and safety in ocular therapeutic agents. However, one noted challenge associated with CA membranes has been a propensity for fouling, which can lead to reduced filtration efficacy over time. The fouling typically results from relatively low surface hydrophilicity, which can allow accumulation of biological matter when used with solutions that have not been optimized to prevent it.

Nanoparticles have been incorporated in membrane fabrication as nanofillers for enhanced performance relative to traditional polymers. Many types of nanoparticles have been used to modify membranes including zeolite, graphite, silica, and carbon nanotubes (CNTs). These modifications have contributed to greater membrane flux, mechanical, chemical and antifouling properties. Carbon-based nanoparticles have attracted special attention due to their unique and distinctive mechanical properties, flexibility and stiffness, as well as electrical and thermal conductivities with high water treatment efficiency in removal of various chemical and biological contaminants. The dispersion capacity of carbon-based nanoparticles in a variety of polymer matrices such as polyvinylidene fluoride, polystyrene, polyethersulfone, polyacrylonitrile, polyamide, and CA has also facilitated further commercial developments.

Carbon-based nanoparticles, particularly CNTs, have also been shown to remove a wide range of contaminants, including heavy metals, metalloids, and organics when used as an adsorbent media (Baby et al., 2019; Fiyadh et al., 2019; Bassyouni et al., 2020). The excellent adsorbent properties have been attributed to the increased specific surface area, the mesoporous (super-nanoporous) structure, the CNTs negative surface charge, and the CNT and aromatic compounds π-π stacking interaction. These properties make them a convenient choice for polymer composite modification.

Fullerene nanoparticles, with their unique spherical geometry and closed carbon shell, offer interesting possibilities in environmental and technological fields. Fullerenes exhibit a high degree of electron mobility and photophysical properties that facilitates capturing and reacting with free radicals. This characteristic of fullerenes makes these nanoparticles particularly effective in applications requiring antioxidant activity, such as in the degradation of environmental pollutants or in the stabilization of materials against oxidative damage. Of note, fullerenes have the ability to undergo functionalization, which allows for the enhancement of their solubility and interaction with various substrates. Functional groups can be attached to the fullerene structure, facilitating specific interactions with target molecules. This functionalization is key to expanding the range of applications of fullerenes, from delivery systems where fullerenes serve as drug carriers, to sensors and catalysts, where fullerenes can interact selectively with specific chemicals.

U.S. Pat. No. 10,123,904B2 entitled, Preservative Removal from Eye Drops, describes a device for removing preservatives from eye drops, featuring a plug made from hydrophilic polymeric gel microparticles. These particles swell upon contact with eye drop solutions, selectively absorbing preservatives like BAK. The device is integrated into multi-dose dispensers, ensuring the delivery of preservative-free medication upon administration. In contrast, this proposed patent utilizes functionalized fullerenes bound within a CA membrane to remove such preservatives in medical dispensing formats including but not limited to eye care therapeutics.

PCT WO2022031841A1 entitled, Systems and Methods for Preservative Removal from Ophthalmic Formulations, addresses the selective removal of preservatives from ophthalmic solutions without altering the concentration of the therapeutic agents. The patent introduces a polymeric matrix designed to absorb preservatives while passing the therapeutic solution to ensure consistent dosage. The polymeric matrix includes both active and inactive components, where the active component may include materials like polyvinyl alcohol or polyacrylamide hydrogels. Whereas the inactive component is generally a polyolefin, which does not interact with the ophthalmic agent or the preservative, allowing for efficient preservative removal, ensuring minimal residue and maintaining the efficacy of the ophthalmic agent at high levels post-filtration. The proposed patent differs from WO2022031841A1 by utilizing functionalized fullerenes to target and remove preservatives before administration without the use of complex polymeric structures.

PCT WO2023201315A2, entitled, Ophthalmic Agent in Preservative Removal Device, outlines methods and devices for dispensing ophthalmic formulations that simultaneously remove preservatives while ensuring that the efficacy of the ophthalmic agent is maintained. WO2023201315A2 features a polymeric matrix that selectively removes the preservative when the formulation is dispensed, allowing for safe multi-dose usage without refrigeration. The matrix ensures that the activity of hydrophobic ophthalmic agents (i.e., latanoprost or timolol) are preserved, typically achieving intraocular pressure reduction. The system is designed to maintain high activity levels of the ophthalmic agent while minimizing preservative levels after passing through the matrix.

In contrast to the latter and other prior art cited herein, the proposed patent differs with respect to, (1) material composition: the novel inclusion of specifically functionalized fullerenes bound to a CA-membrane to selectively remove preservatives from ophthalmic solutions. This contrasts from the use of polymeric hydrogels, such as crosslinked polyacrylamide modified with different monomers. Further, (2) the method of action in the proposed patent leverages the chemical properties of fullerenes to capture and remove preservatives, as opposed to the sieving action of the polymeric matrices used in the prior art. Finally, (3) the application flexibility of the proposed invention allows for customization to target specific preservatives, offering potentially greater efficiency and specificity in purification. These differences offer more targeted and efficient preservation of the ophthalmic agent integrity and efficacy through the use of nanotechnology.

As a primary component of the present invention, fullerenes are characterized by their unique spherical shapes and cage-like structures. Fullerenes represent a novel class of carbon allotropes that have influenced advancements in materials science and biotechnology within various medical fields. These nanomaterials consist of carbon atoms linked by single and double bonds to form closed or partially closed meshwork cages, with each carbon atom hybridized in the sp2 state. The electronic properties of fullerenes stem from their extensive π-bond conjugation and distinct p-orbitals, which facilitate electron reception and scavenging abilities, making them suitable for a range of applications, especially in medicine due to their biocompatibility. A key attribute associated with fullerenes is the versatility to modify their surface properties through functional groups and encapsulate molecules inside of the carbon cages. The structural integrity of fullerenes, characterized by a spherical arrangement of planar benzene rings, places unique constraints on the π-electron orbitals. This configuration enables remarkable electron transfer capabilities, attributable to their low reorganization energy and the presence of low-lying excited states, as both singlets and triplets.

Carbon nanoparticles, including CNTs, nanodots, fullerenes, etc., have been previously used as detectors for mercury. The fullerenes provide high surface area, high reactivity (due to the π-π interactions) and the versatility for functionalization to target specific molecules, as above. Fullerene nanoparticles functionalized with thiol groups for different applications and sulfur have been used in many fields, particularly in mercury sensing, due to its photostability. However, using sulfur nanoparticles can decrease activity due to the larger particle size. One solution to this issue has been to use sulfur nanodots, which are relatively small and offer a larger surface area, which can facilitate more activity.

### SUMMARY OF THE INVENTION

This proposed invention advances the safety and effectiveness of ophthalmic and medical treatments by introducing a filtration system to selectively remove preservatives from medical solutions. The filter is adaptable to various dispensing systems and would preserve the antimicrobial properties essential for multi-dose formulations while safeguarding patients from various additives. This innovation can enhance patient safety as well as broaden the use of essential preservatives in biomedical products without compromising safety, thereby establishing a new standard in the delivery of ophthalmic and other medical care.

The primary beneficiaries of this patent include eye disease patients, treatment specialists, engineers, and educators, who collectively drive the development of ophthalmic solutions for eye care. This invention also addresses the urgent need for effective and affordable eye care treatments, which are currently in global short supply.

The present invention introduces a filtration system that adsorbs and removes mercury-containing, quaternary ammonia, zinc core preservatives and antimicrobial compounds, and SOCs from ophthalmic and other medical solutions using targeted fullerene derivative-based filters. This patent introduces a functionalized fullerene (fullerene derivatives) modified cellulose acetate (CA)-based filtration system. For the purposes of this patent, the terms “fullerene,” “fullerene derivative,” and “functionalized fullerene” are used interchangeably. Each of these terms refers to a modified fullerene, which is a fullerene structure that has been chemically altered through the addition of functional groups or other chemical modifications to enhance its properties and functionality. For the purposes of this patent, the terms adsorption and absorption are also used to describe distinct but simultaneous processes occurring within the material. Adsorption refers to the process where molecules adhere to the surface of the CA membrane or embedded nanoparticles via physical or chemical interactions. Absorption, on the other hand, describes the uptake and distribution of fluids into the bulk of the CA matrix. These processes work synergistically to enhance the filtration efficiency and removal of preservatives and contaminants from the solution.

The invention provides a modular design that can be integrated into dropper bottles and dispensing appliances for ophthalmic solutions, as well as broader utility in other medical applications to ensure sterility and safety. The proposed filtration system removes, adsorbs and selectively scavenges mercury-containing (i.e., thimerosal) and quaternary ammonia (i.e., BAK) preservatives, as well as “vanishing” or “soft” SOCs (i.e., Purite™) and ionic buffered (i.e., Sofzia™) preservatives prior to administration, mitigating their potentially toxic effects. By removing these preservatives immediately before a solution is administered, the invention ensures the antimicrobial benefits during storage, while eliminating preservative exposure for the user during application.

A key component associated with the filtration system of this patent is the inclusion of nanoparticles into the polymer matrix of the modified CA membrane. The inclusion of both organic and inorganic nanoparticles can further enhance the mechanical stability, thermal stability and filtration efficiency of membranes, as well as contribute to the physical properties and functional capabilities of the membrane, allowing for more precise and selective targeting of specific contaminants, including preservatives used in ophthalmic solutions.

In this invention, the introduction of amine-functionalized CA polymers that bind with specifically and chemically functionalized fullerenes can overcome fouling dynamics and provide a targeted system. Incorporation of fullerenes into CA has been shown to enhance flux (highest amongst carbon-based nanoparticles), allowing for a greater flow rate of substances passing through the membrane (Kuzminova et al., 2024). Likewise, rejection coefficients, or the membrane capacity to filter out unwanted particles were found to improve with the inclusion of fullerenes (Kuzminova et al., 2024). However, the flux recovery ratio, which is a measurement of the membrane capacity to return to its original flux rate after being fouled and then cleaned, was shown to decrease with the addition of fullerenes (Kuzminova et al., 2024). In the present invention, the functionalized fullerene adsorption of preservative compounds onto the surface of the filtration system establishes an antimicrobial and anti-biofouling membrane with enhanced performance and longevity attributes.

The filtration system described herein utilizes targeted functionalized fullerenes that are chemically attached to an amine-modified CA membrane. This filtration membrane enhancement is optimized for medical applications, particularly for the targeted removal of mercury-based and quaternary ammonia antimicrobial preservatives, microbes and pathogens, and soft or vanishing preservatives from ophthalmic solutions, as well as other liquids or viscous medical formulations. This invention is compatible with myriad dispensing pathways, such as the neck of a dropper bottle or the outlet of a syringe to extend applications to other medical solutions. The filter system represents a platform to ensure that products such as eye drops, nasal sprays, and injectables are sterile and free from compounds that could cause irritation or toxicity when administered.

The economic potential of the present invention, using advanced materials at the atomic scale, would alter traditional chemical engineering paradigms regarding bulk raw material utilization. For instance, the manufacturing supply of the novel filtration agents could reduce physical production requirements. Secondarily, the ability to safely integrate thimerosal into ophthalmic solutions would facilitate traditional, at-scale bulk production that could be stored safely over extended periods. This approach could maximize manufacturing efficiency, reduce environmental waste, and minimize the cost of goods manufactured relative to unit-dose and preservative-free formulations.

### BRIEF DESCRIPTION OF THE DRAWINGS

FIG. 1 is a molecular representation of an exemplary microfilter membrane composed of amine functionalized CA and thiolated-fullerene of 60 carbons functionalized with thiol (SH) containing sulfur nanodots (gray circle) encapsulated and/or attached to the fullerene cage.

FIG. 2 is a molecular representation of an exemplary microfilter membrane composed of amine functionalized CA and halo-fullerene of 60 carbons functionalized with iodide (I) containing sulfur nanodots (gray circle) encapsulated and/or attached to the fullerene cage.

FIG. 3 is a molecular representation of an exemplary microfilter membrane composed of amine functionalized CA and EDTA-fullerene of 60 carbons functionalized with EDTA containing sulfur nanodots (gray circle) encapsulated and/or attached to the fullerene cage.

FIG. 4 is a molecular representation of an exemplary microfilter membrane composed of amine functionalized CA, sulfur nanodots (gray circle) and EDTA-fullerene of 60 carbons functionalized with EDTA.

### DETAILED DESCRIPTION OF THE INVENTION

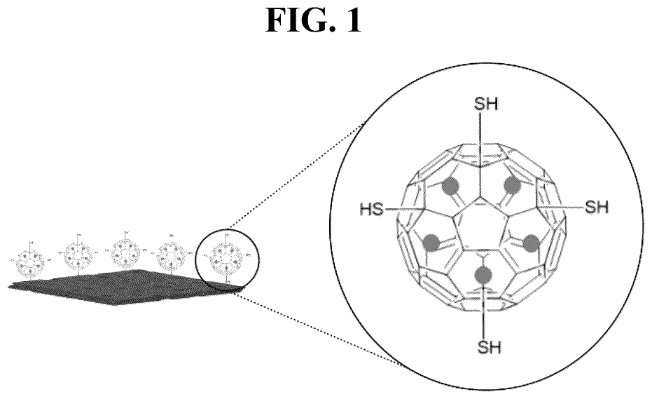

In the present invention, a fullerene derivative adsorption trap would prevent patient exposure to undesirable therapeutic additives, such as preservative molecules. Moreover, the nanoparticle trap would be fabricated onto MF CA membranes (FIG. 1) with dimensions of 0.1-1.0 μm using amine functionalized CA membranes.

In the primary embodiment, membrane preparation of the fullerene derivative-amine functionalized CA nanocomposite membrane is accomplished via a phased inversion technique: 1) The CA is first functionalized with an amine group in a two-step reaction: CA is dissolved in DMSO at 40° C. before the addition of an activating agent (e.g., an electrophile such as benzoquinone) to provide an active site. 2) 1,2,3-triamine propane is then added at 40° C. and stirred for 4-hours to replace the benzoquinone with the amine. 3) Following that, fullerenes are dispersed by ultrasonication in chloroform using a probe sonicator (200 W) for 1 hour to achieve a uniform distribution. 4) Next, a solution of the amine functionalized CA is prepared by dissolving it in acetone under stirring conditions to create a homogeneous mixture. 5) Once the fullerene dispersion is thoroughly mixed, it is added to the CA solution, and the combined solution is stirred continuously for 24-hours at 60° C. to ensure complete integration of fullerenes into the CA matrix and to enhance polymer chain integration and composite stability. 6) The resultant casting solution is then degassed by ultrasonication for 1 hour to remove any trapped ambient air, ensuring a smooth film. 7) The degassed mixture is then cast onto a glass plate using a knife blade applicator set to 250 μm thickness. 8) The cast film is immediately immersed in a water bath to cast the CA membrane. The process is conducted at room temperature to facilitate the phased inversion process. 9) The membranes are then rinsed with deionized water to remove solvents and additives and stored in deionized water after complete precipitation.

The amine functionalized CA membranes with fullerene derivatives incorporated into the filter matrix in the present invention are engineered to provide hydraulic permeability, allowing for dispensing of ophthalmic and similar solutions through basic dropper bottles with minimal physical effort. The hydraulic permeability of the membrane is a critical aspect of its design, influencing the ease of fluid passage and the specificity of targeting the unwanted contaminant and/or preservative removal, without affecting the concentration of active ingredients of the administered therapeutic solution.

One embodiment of the present invention comprises a filtration system that is specifically designed to eliminate mercury-containing preservatives from ophthalmic and other medical solutions. These integrated amine-functionalized CA MF membranes are embedded with thiol-functionalized fullerenes encapsulated with sulfur nanodots (FIG. 1). These components selectively trap and remove mercury-containing compounds due to the thiophilicity of mercury. This filter is designed to reduce the risks associated with mercury toxicity while retaining the preservative's antimicrobial activity in the solution during storage. These membranes can also scavenge and remove soft preservatives without interacting with the active ingredients of the respective solutions.

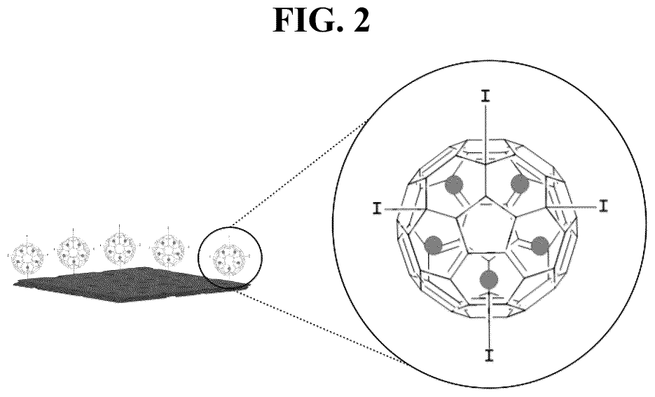

Another aspect of the present invention comprises a filtration system that is designed to eliminate quaternary ammonia preservatives from ophthalmic and other medical solutions. This embodiment of the present invention comprises MF CA membranes embedded with iodine-functionalized fullerenes encapsulated with sulfur nanodots (FIG. 2). These elements are specifically engineered to capture and eliminate BAK from solutions immediately prior to administration. This filter is designed to reduce the risks associated with BAK toxicity while retaining the preservative's antimicrobial activity in the solution during storage. The binding process exploits the lone pair of electrons in the iodine atom of the functional group on the fullerene, which interacts with the positively charged nitrogen in the BAK quaternary amine structure. Additionally, the inclusion of sulfur nanodots enhances the membrane performance by providing additional active sites, utilizing a similar mechanism where the lone pair of electrons in sulfur contributes to the interaction.

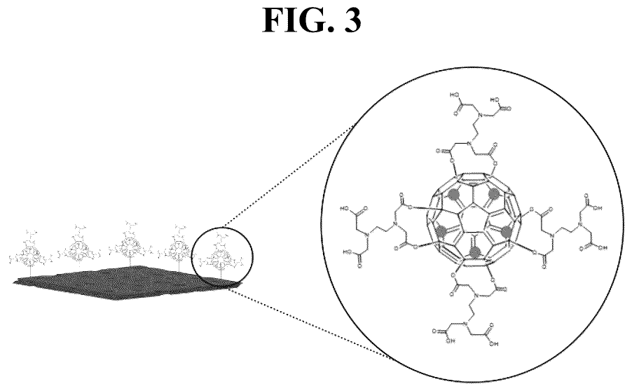

A third embodiment of the present invention comprises MF CA membranes embedded with EDTA-functionalized fullerenes encapsulated with sulfur nanodots (FIG. 3). EDTA is a pharmaceutical substance used as an antidote for heavy metal toxicity; it is also commonly used for anticoagulation for laboratory plasma specimens, as well as in the beauty and food and beverage industries. EDTA is classified as a chelating drug, and it binds to metal ions (e.g., mercury). Notably, EDTA also interacts with quaternary ammonium compounds. The components in this embodiment are thus designed to trap and remove a wide range of preservative compounds, as this functionalized fullerene can remove preservatives through different mechanisms including chelation, ionic bonding, hydrogen bonding, physical adsorption, electrostatic interactions, size exclusion or sieving, catalytic decomposition, and complexation.

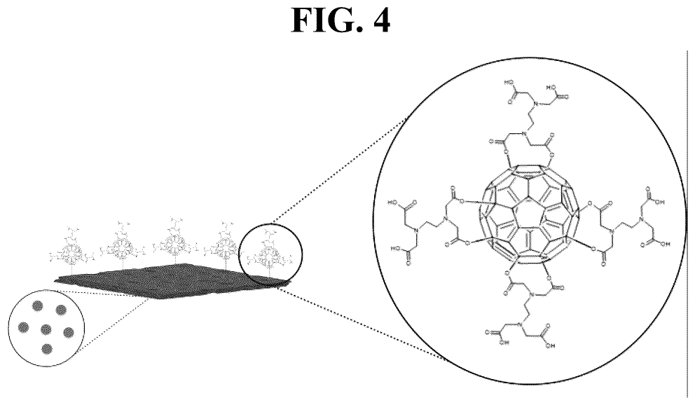

A fourth embodiment of the present invention comprises MF CA membranes embedded with sulfur nanodots and EDTA-functionalized fullerenes (FIG. 4). In this embodiment, more active sites are available to provide an enhanced trap. These components are designed to adsorb, trap and remove a range of preservative compounds.

The composition of the present invention provides the structural benefits of MF membrane technology, as well as specifically targeting soft or disappearing preservatives. Another embodiment of the present invention would remove SOCs and zinc, borate, propylene glycol, and sorbitol-containing ionic buffered preservatives. The amine groups in the polymer not only facilitate water flow, they also chelate with the zinc in zinc-containing ionic buffered preservatives, enhancing filtration efficiency. The thiophilicity of the zinc provides another mechanism for adsorption at the surface of the sulfur nanodots. The thiol groups, which would be functionalized onto the surfaces of fullerene cages and incorporated with the CA, are also designed to target SOC preservatives. Thiol groups are effective in reacting with and capturing SOCs by-products, which include various reactive oxygen species.

The filter system of the present invention is designed for integration into dropper bottles, syringes or other medical or industry dispensers and may be comprised of a single phase membrane or a multi-stage filter to provide broader adsorption. Additionally, the ability to use multiple antimicrobials or preservatives at greater concentrations may be beneficial in combating the rise of antimicrobial-resistant (AMR) pathogens. Another aspect of the present invention is associated with the removal of microbial contaminants. While the inclusion of preservative materials and broad-spectrum antimicrobials in medical solutions would lead to greater material sterility, the need to remove intact, potentially resistant pathogens would be especially advantageous in an era of antimicrobial resistance. The CA MF membranes are categorized as low-pressure membranes that use physical separation to remove pathogens, including bacteria, yeast cells, and organic colloids. Combined with inclusion of engineered fullerene derivatives, the membrane system would be capable of removing preservatives that adhere to the fullerenes, as well as pathogens. In addition, the ability for CA to be fabricated in different types of membranes provides more flexibility to use this invention in medical solutions based on their composition (i.e., active agents or agent molecular size) such that the membrane can be fabricated in the optimal pore size.

The membrane in the primary embodiment would also provide hydraulic permeability such that relatively low pressure is required to dispense the fluid. This property ensures that the active ingredients of the solution can be administered by forming a drop at the tip of the dropper without delay or perceptible resistance. However, in order to enhance the efficacy of these membranes in adsorbing the preservatives, thicker membranes could be fabricated to provide more stationary surface contact for adsorption. The hydraulic permeability of the membrane depends significantly on the pore size engineered into the matrix. Larger pores may allow for higher flow rates for a given pressure drop across the membrane, facilitating the administration of the solution regardless of the viscosity of the formulation, as in rewetting eye drops.

In certain embodiments, the hydraulic permeability of the membranes may be greater than about 0.01 Darcy (Da). For instance, a nozzle part of the dropper may comprise a permeability of approximately 0.1 Da. Where hydraulic permeability ranges from 1 to 10 Darcy, the fluid may be retained in the filter when the pressure may be lowered following the formation of a drop. This facilitates the prevention of backflow or leakage and can help ensure correct dosage with each use. A higher hydraulic permeability, above 10 Da, 100 Da, or up to 1,000 Da, would allow the same membrane to be utilized across a range of formulations. This adaptability is important for ophthalmic solutions with varying viscosity and compositional characteristics. The porous polymeric matrix of the membrane, enhanced with nanofillers, would be specifically tailored to have hydraulic permeability values from 0.01 Da, to 0.1 Da, 1 Da, 10 Da, or to 100 Da, or within a range defined by any two of these values, providing flexibility and utility in medical applications.

In the primary embodiment, treatment of the membrane with nanofillers would facilitate the passage of therapeutic active ingredients while ensuring that targeted components are adsorbed and filtered out, such as mercury-based and quaternary ammonia preservatives, soft/disappearing preservatives and microbial matter.

To further enhance the functionality of the embodiment for a filtration system for ophthalmic applications, the functionalized membrane may be coated with an ultrahydrophobic layer. This coating would be applied to prevent the ophthalmic solution from coming into unintended contact with the membrane during storage, whereby the solution might otherwise remain in prolonged contact with the membrane due the positioning of the container. The ultrahydrophobic layer would repel the solution, thus preserving the integrity of the therapeutic agents and allow preservative absorption only from the administered volume of the solution at the time of administration. However, the utility of this ultrahydrophobic coating is designed such that it does not hinder the dispensing functionality of the dropper when pressure is applied. Upon squeezing the dropper bottle, the pressure would overcome the hydrophobic barrier, allowing the solution to penetrate the CA membrane. Once in contact with the membrane, the solution would be effectively processed—unwanted components would be filtered out while the desired ingredients pass through.

The present invention comprises a functionalized fullerene and CA-amine membrane-based filter designed to remove biocidal preservatives from ophthalmic therapeutic solutions. It would be obvious to someone skilled in the art that the filter composition of this invention would be applicable for other medical solutions such as nasal sprays, injectables, or in general, any solution that requires sterility with multiple use or dosage—and which could thereby maximize dating with safe inclusion of proven preservatives removed at the point of administration.

What is claimed:

1. A filtration system for ophthalmic and medical solutions, comprising one or more:
a. A microfiltration membrane consisting essentially of an amine-functionalized cellulose acetate and thiolated-fullerene containing sulfur nanodots encapsulated and/or attached to the fullerene cage;
b. A microfiltration membrane consisting essentially of an amine-functionalized cellulose acetate and iodinated-fullerene containing nanodots encapsulated and/or attached to the fullerene cage;
c. A microfiltration membrane consisting essentially of an amine-functionalized cellulose acetate and EDTA-fullerene containing sulfur nanodots encapsulated and/or attached to the fullerene cage; and
d. A microfiltration membrane consisting essentially of an amine-functionalized cellulose acetate.

2. The filtration system of claim 1, wherein the thiolated-fullerene derivatives are specifically engineered to trap and remove mercury-containing preservatives, such as thimerosal, through sulfur-mercury bonding.

3. The filtration system of claim 1, wherein the iodinated-fullerene derivatives interact with quaternary ammonium compounds through ionic bonding between the iodine atoms and the positively charged nitrogen in the quaternary amine structure are engineered to trap and remove such compounds.

4. The filtration system of claim 1, wherein the EDTA-fullerene derivatives are effective in chelating metal ions, thereby engineered to trap and remove heavy metal-based preservatives from the solution.

5. The filtration system of claim 1, wherein the sulfur nanodots provide additional active sites for interaction with preservative compounds, enhancing the overall performance of the membrane engineered to trap and remove specific compounds.

6. The filtration system of claim 1, wherein the amine-functionalized cellulose acetate microfiltration is engineered to provide stability, durability and remove microbial contaminants in addition to preservative compounds, thereby enhancing the safety of the ophthalmic solution upon administration.

7. The filtration system of claim 1 that is engineered to trap and remove zinc core preservatives, SOCs, and ionic buffered preservatives from ophthalmic and other medical solutions.

8. The filtration system of claim 1 that is integrated into dropper bottles for ophthalmic and other medical solutions.

9. The filtration system of claim 1 that is integrated into syringes for medical solutions.

10. The filtration system of claim 1, wherein the membrane is engineered to trap and remove preservatives immediately prior to administration, ensuring antimicrobial efficacy during storage without exposing users to potentially toxic effects.

11. The filtration system of claim 1, wherein the microfiltration membrane displays hydraulic permeability, allowing for effective dispensing of ophthalmic and other medical solutions with minimal physical effort.

12. The filtration system of claim 1 that is coated with an ultrahydrophobic layer to prevent contact with the solution during storage, engineered only to trap and remove preservatives immediately prior to administration.

13. The filtration system of claim 1 that is a single filter system or a multi filter system to provide broader adsorption capacity.

14. The filtration system of claim 1, wherein the amine-functionalized cellulose acetate microfiltration membrane has an average pore radius of 0.1-1.0 μm.

15. The filtration system of claim 1, wherein the membrane is engineered to trap and remove multiple antimicrobials or preservatives at higher concentrations as may be required to mitigate the risks from antimicrobial-resistant pathogens.

16. The filtration system of claim 1, wherein the sulfur nanodots provide increased surface area and enhanced reactivity to trap and remove specific compounds.

17. The filtration system of claim 1, wherein the filtration system maximizes the shelf life and safety of stored medical solutions by removing preservatives prior to use, while retaining their antimicrobial benefits during storage.

18. The filtration system of claim 1, which could be formulated with different properties, including the pore size such as UF, NF, RO, FO.

19. The filtration system of claim 1 engineered with any type of porosity that is integrated but not limited to dropper bottles for ophthalmic solutions.
